(19) United States
(12) Patent Application Publication
(10) Pub. No.: US 2021/0220400 A1
(43) Pub. Date: Jul. 22, 2021

(54) INHIBITION OF CANDIDA AURIS BIOFILM FORMATION ON MEDICAL AND ENVIRONMENTAL SURFACES BY SILVER NANOPARTICLES

(71) Applicant: Board of Regents, The University of Texas System, Austin, TX (US)

(72) Inventors: Humberto Herman Lara VILLEGAS, San Antonio, TX (US); Jose L. LOPEZ-RIBOT, San Antonio, TX (US); Miguel JOSE-YACAMAN, San Antonio, TX (US)

(21) Appl. No.: 17/145,056

(22) Filed: Jan. 8, 2021

Related U.S. Application Data
(60) Provisional application No. 62/958,637, filed on Jan. 8, 2020.

(51) Int. Cl. A61K 33/38; A61K 9/70; A61P 31/04; A61P 31/10; A61P 31/12; A61L 31/16; A61L 31/06

(52) U.S. Cl. CPC A61K 33/38; A61K 9/7007; A61P 31/04; A61P 31/10; A61L 2400/12; A61L 31/16; A61L 31/06; A61L 2300/104; A61P 31/12

(57) ABSTRACT

Materials, compositions, and methods of inhibiting biofilm formation and treating formed biofilm employing medically acceptable materials and silver nanoparticles coupled thereto are disclosed. Pathogenic fungi, bacteria, viruses, and combinations thereof are implicated in healthcare-associated infections with both significant medical consequences and high mortality rates. Substantially pure silver nanoparticles are coupled directly to polymer or textile medical dressings to treat fungal, bacterial, viral and combination infections.

### BACKGROUND OF THE INVENTION

Results. Substantially pure round AgNPs (1 to 3 nm in diameter) were formed using a microwave-assisted synthetic approach. When tested against C. auris, the results indicated a potent inhibitory activity both on biofilm formation (IC₅₀ of 0.06 ppm) and against preformed biofilms (IC₅₀ of 0.48 ppm). Scanning Electron Microscopy (SEM) images of AgNP-treated biofilms showed cell wall damage mostly by disruption and distortion of the outer surface of the fungal cell wall. In subsequent experiments AgNPs were used to functionalize medical and environmental surfaces. Silicone elastomers functionalized with AgNPs demonstrated biofilm inhibition (>50%) at relatively low concentrations (2.3 to 0.28 ppm). Bandage dressings loaded with AgNPs inhibited growth of C. auris biofilms by >80% (2.3 to 0.017 ppm). Also, to demonstrate long lasting protection, dressings loaded with AgNPs (0.036 ppm) were washed thoroughly with PBS, maintaining protection against the C. auris growth from cycles 1 to 3 (>80% inhibition), and from cycles 4 to 6 (>50% inhibition).

Dose-dependent activity of silver nanoparticles (AgNPs) against biofilms formed by fungi, bacteria, and combinations thereof, including, for example, C. auris on both medical (silicon elastomer) and environmental (bandage fibers) surfaces and combined C albicans/methicillin resistant Staphylococcus aureus (MRSA) on biomedical surfaces. Further, the AgNP functionalized materials are thought to exhibit antiviral activity, for example, against SARS-CoV The AgNPs-functionalized fibers retain the fungicidal effect even after repeated thorough washes. Overall these results point to the utility of silver nanoparticles to prevent and control infections cause by emerging pathogenic fungi, bacteria, and viruses.

### SUMMARY OF THE INVENTION

In recent years systemic fungal infections caused by Candida auris have been reported and are rapidly spreading to different parts of the world. This newly described species, closely related to C. haemulonii in the Metschnikowiaceae clade, was first described in Japan in 2009 as an emerging multidrug-resistant (MDR) ascomycetous yeast pathogen. It causes BSIs associated with high mortality. The transmission of C. auris is a recognized risk in health-care settings leading to widespread and difficult to control health care-associated infections (HAIs) outbreaks. To further complicate matters, unlike other Candida sp., C. auris survives and proliferates for weeks either on dry or moist surfaces in health-care facility settings. Although normally unable to form hyphae, C. auris yeasts can develop large aggregates of cells and biofilms embedded by an extracellular polymeric substance (EPS) matrix that effectively shield them against external harmful factors; mainly against the hosts immune responses and antifungal drugs. Drug resistance of C. auris to all three major classes of commonly antifungal medical treatments (polyenes, equinocandins and azoles) has been described and this is a major limitation to achieve effective antifungal therapy, which is further amplified by the formation of biofilms. Therefore. there is an urgent need to discover new effective antifungal strategies to combat this emerging MDR pathogen and stop the HAIs outbreaks.

Silver as a medical treatment had been used for many years and was the most commonly used broad-spectrum antibacterial compound before the discovery of antibiotics in the early 20th century. Even today, silver sulfadiazine (SSD) cream, is widely used to treat wound infections and in burn therapy. Most recently, advances in nanotechnology have emerged with significant potential impact for the treatment of drug-resistant infections. Application of silver nanoparticles (AgNPs) for health care environments has attracted international attention, since these nanomaterials could be used in the fight against multidrug resistant (MDR) organisms and HAIs. It has previously been reported on the potent activity of positively charged AgNPs against methicillin resistant Staphylococcus aureus (MRSA) and Candida albicans biofilms. It is mostly believed that the attachment of AgNPs to the surface leads to the disruption of the cell membrane integrity, permeabilizing the cell wall/membrane and inducing apoptotic cell death.

Thus, considering the fungicidal properties of AgNPs and the interest and urgent need to control the spread of C. auris infections in health care settings, here the present disclosure identifies potent activity of substantially pure AgNPs synthesized by microwave-assisted techniques against C. auris, with emphasis on the inhibition of C. auris biofilm formation on biomedical and environmental surfaces functionalized with AgNPs.

The present disclosure also pertains to the activity of substantially pure AgNPs against bacterial infection and the inhibition of bacterial biofilm formation on biomedical and environmental surfaces functionalized with AgNPs.

The present disclosure further pertains to the activity of substantially pure AgNPs against viral infection and the inhibition of viral infections

Finally, the present disclosure pertains to activity of substantially pure AgNPs against combined fungal/bacterial infection and inhibition of combined fungal/bacterial biofilm formation on biomedical and environmental surfaces functionalized with AgNPs.

### BRIEF DESCRIPTION OF THE FIGURES

FIG. 1A is a dose-response curve of inhibition of C. auris biofilm formation by AgNPs at two-fold serial dilutions. IC₅₀ values for inhibition of biofilm formation were calculated as AgNPs 0.06 ppm

FIG. 1B is a dose-response curve of activity of AgNPs against preformed C. auris biofilms. The IC₅₀ for pre-formed biofilm inhibition was calculated as AgNPs 0.48 ppm.

FIG. 2 are scanning electron micrographs visualizing the effects of silver nanoparticles on C. auris biofilms. Panels a, c, and e are SEM micrographs of a preformed biofilm of C. auris without treatment. Panels b, d, and f are SEM micrographs of a preformed biofilm after AgNPs treatment (0.48 ppm).

FIG. 3 is an Energy Dispersive X-Ray Spectroscopy (EDS) spectra of silver demonstrating the presence of elemental silver signal on the surface of silicone elastomer sheets (SES) functionalized with silver nanoparticles. An SEM micrograph insert shows dots in the SES that indicate silver signal detection by spectral mapping acquisition.

FIG. 4A is a dose-response curve of inhibition of C. auris biofilm growth on SES.

FIG. 4B are SEM micrographs showing C. auris biofilm formation on the surface of SES without AgNPs in panel b; the SES surface in the absence of C. auris and silver nanoparticles in panel c, and functionalization of SES with AgNPs (0.48 ppm) showing inhibition of C. auris biofilm formation after 24 h incubation at 37° C.

FIG. 5 is a composite SEM micrograph showing two magnifications of elastic bandage wraps (EBW) fibers with AgNPs attached to the fiber surface and after washing with PBS.

FIG. 6A is a graph showing inhibition of C. auris biofilm formation on EBW functionalized by incubation with different concentrations of AgNPs.

FIG. 6B are SEM micrographs confirming biofilm inhibitory activity of functionalized EBWs with panels b and c showing EBW fibers without AgNPs or C. auris as negative control; panels d and e showing EBW fibers with C. auris growth as positive control; and panels f and g showing EBW Fibers functionalized with AgNPs attached to the surface inhibited C. auris growth.

FIG. 7 is a graph showing levels of inhibition of C. auris biofilm formation on EBWs functionalized with AgNPs after cycles of PBS washes with PBS every 24 h for eight consecutive days.

FIG. 8 is a dose-response curve demonstrating inhibitory activity of AgNPs against mixed C. albicans/Methicillin-resistant Staphylococcus aureus (MRSA). The IC₅₀ was calculated as AgNPs 0.53 ppm.

FIG. 9 is are four SEM micrographs visualizing the effects of AgNPs against dual-species biofilms of C. albicans and MRSA. Panels (a) and (c) are images of preformed mixed biofilms without AgNPs treatment showing well-defined fungal (arrow 1) and MRSA (arrow 2) cells with a smooth surface, oval shaped, and showing abundant extracellular polymeric substances of the biofilm (arrow 3). Panels (b) and (d) illustrate changes on the preformed mixed biofilm after 24 hour treatment AgNPs (0.53 ppm)(arrow 4) presenting alteration, and disruption on the C. albicans yeast outer cell membrane (arrow 5) and fewer MRSA cells (arrow 6).

FIG. 10 are an SEM micrograph (panel a) and an EDS spectra (panel b) of silver nanoparticles on the surface of the functionalized silicone elastomers sheets. Red spots pinpoint Ag+ detection by spectral (panel a) and mapping acquisition in the silicone elastomer (panel b). The blue arrow points to the detection of a strong silver (Ag) signal.

FIG. 11 is a dose-response graph illustrating inhibition of mixed biofilm growth on functionalized silicone elastomer sheets.

FIG. 12 are opto-digital microscopy 2D images showing inhibition of mixed biofilm by AgNP functionalized silicone elastomers illustrating inhibition of dual-species C. albicans/MRSA biofilms: visualization using opto-digital microscopy. Panel a shows a mixed biofilm and growth inhibition, panel B shows a non-functionalized silicone elastomer without biofilm growth as negative control showing the rugged surface of the elastomer, panel c shows mixed biofilm growth on non-functionalized silicone elastomer showing true hyphae, MRSA, yeast cells, and extracellular polymeric substances (EPS).

### DETAILED DESCRIPTION OF THE DISCLOSURE

The scope of the disclosure is accordingly to be limited by nothing other than the appended claims, in which reference to an element in the singular is not intended to mean “one and only one” unless explicitly so stated, but rather “one or more.” It is to be understood that unless specifically stated otherwise, references to “a,” “an,” and/or “the” may include one or more than one and that reference to an item in the singular may also include the item in the plural. All ranges and ratio limits disclosed herein may be combined.

Moreover, where a phrase similar to “at least one of A, B, and C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching when used throughout the figures to denote different parts but not necessarily to denote the same or different materials.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component; region, layer or section without departing from the teachings of the example embodiments.

Spatially relative terms, such as “inner,” “outer,” “beneath,” “below.” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over; elements described as “below”, or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

“Substantially” is intended to mean a quantity, property, or value that is present to a great or significant extent and less than or equal to totally.

“About” is intended to mean a quantity, property, or value that is present at ±10%. Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. In addition, disclosure of ranges includes disclosure of all values and further divided ranges within the entire range, including endpoints given for the ranges.

The steps recited in any of the method or process descriptions may be executed in any order and are not necessarily limited to the order presented. Furthermore, any reference to singular includes plural embodiments, and any reference to more than one component or step may include a singular embodiment or step. Elements and steps in the figures are illustrated for simplicity and clarity and have not necessarily been rendered according to any particular sequence. For example, steps that may be performed concurrently or in different order are illustrated in the figures to help to improve understanding of embodiments of the present disclosure.

Any reference to attached, fixed, connected or the like may include permanent, removable, temporary, partial, full and/or any other possible attachment option. Additionally, any reference to without contact (or similar phrases) may also include reduced contact or minimal contact. Surface shading lines may be used throughout the figures to denote different parts or areas but not necessarily to denote the same or different materials. In some cases, reference coordinates may be specific to each figure.

Systems, methods, and apparatus are provided herein. In the detailed description herein, references to “one embodiment,” “an embodiment,” “various embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Turning now to the description of the disclosure, there is disclosed a composition, method and use of the composition described for treating fungal, bacterial, viral and combination infections using substantially pure silver nanoparticles directly coupled to a medical-grade substrate, such as silicone elastomer or medical textiles, such as elastic bandage wraps.

As used herein, the identified fungi, bacteria, viruses, or combinations thereof are intended to be exemplary and non-limiting to the scope of the invention. Those skilled in the art will understand and appreciate that the methodologies employed and disclosed herein may be used to identify the inhibitory activity of fungus, bacterium, virus, or combination infections for other non-specified pathogenic organisms.

Inhibition of C. auris Biofilm: Materials and Methods

All chemicals used in this study were purchased from Sigma-Aldrich (St. Louis, Mo., USA), unless otherwise stated.

Medical-grade Silicone elastomer sheets (SES) were obtained from Bentec Medical (Woodland, Calif., USA). The elastic bandage wrap (EBW) was obtained from Life Wear Technologies Inc. (Lighthouse Point, Fla., USA).

### Preparation and Characterization of AgNPs

Pure AgNPs were synthesized through a microwave (MW) irradiation-assisted heating reaction using an Ethos EZ® Digestion System microwave (Milestone, Inc.; Shelton, Conn., USA) as described before.^{19,20} Briefly, 1.7 g of AgNO₃ were dissolved in 10 ml of distilled water (DI) and treated by MW irradiation. The AgNO₃ solution was continuously irradiated for 15 seconds at 1000 W. After MW-irradiation, samples were cooled down at room temperature. The characterization of the physicochemical properties of the resulting AgNPs was performed. The final concentration of the colloidal solution after MW-irradiation was determined using a double-beam atomic absorption spectrophotometer (AA-6200, Shimadzu Corporation, Kyoto, Japan). Zeta potential (ZP) was measured to determine the surface charge of AgNPs in solution at 25° C. using the Zetasizer Nano ZS (Malvern Instruments Ltd, Malvern, Worcestershire, UK) and also to demonstrate the electrokinetic potential and the colloidal stability of the AgNPs. The ζ-potential value of the particle surface charge increased from −2.9 to +13.4 mV over a 120 h time period. ζ-potential shifted towards more positive surface charge; this change confirms the adsorption of cations onto nanoparticles. High-resolution Transmission Electron Microscope (TEM) analysis (JEM-2010, Jeol Ltd., Tokyo, Japan) was used to obtain images of the AgNPs to measure the particle size analysis and shape of the metal AgNPs, indicating that the metallic particles are round in shape, with the average size between 1 to 3 nm (not shown).

### Strains, Media and Culture Conditions

Candida auris 0390 strain was obtained from the Centers for Disease Control and Prevention Antibiotic Resistance Isolate Bank (CDC, Atlanta, Ga., USA).²⁶ This MDR isolate is resistant to amphotericin B, azoles and shows decreased echinocandin sensitivity. Cryopreserved yeast cells stored as glycerol stocks in an ultra-low freezer (set at −80° C.) were propagated by streaking a loopful of yeast cells onto agar plates of yeast-peptone-dextrose (YPD). C. auris 0390 strain was cultured overnight into flasks (150 ml) by inoculating yeast cells in 20 ml of liquid YPD medium at 30° C. in an orbital shaker (Thermo Fisher Scientific, Waltham, Mass., USA) at 180 rpm. Yeast cells were washed with sterile phosphate-buffered saline (PBS) twice after 18 h incubation and the final inoculum size was adjusted by hemocytometer to 1×10⁶/mL for biofilm formation and testing in RPMI-1640 medium with L-Glutamine (Cellgro, Manassas, Va., USA) and buffered with morpholinepropanesulfonic acid (MOPS) at 165 mM and pH 6.9 (Thermo-Fisher Scientific, Waltham, Mass.) (“RPMI”).

### Dose-Response Inhibition of Candida Auris Biofilms by AgNPs.

To assess the activity of AgNPs on C. auris biofilm formation and against preformed biofilms a known phenotypic method was used that was previously developed for Candida biofilm formation on the surface of sterile, tissue culture-treated, flat bottom polystyrene 96 well microtiter plates (Corning Incorporated, Corning, N.Y., USA). Yeast cells collected from overnight cultures were washed in sterile PBS and resuspended at a final cell concentration of 1.0×10⁶ cells/mL in RPMI medium. For assessing inhibition of biofilm formation, yeast cells were added to wells of microtiter plates containing serial dilutions of AgNPs at concentrations ranging from 1.15 to 0.008 ppm. The plates were then incubated at 37° C. for 24 h, carefully washed twice with PBS, and the extent of biofilm formation estimated using the tetrazolium salt (2,3-Bis-(2-Methoxy-4-Nitro-5-Sulfophenyl)-2H-Tetrazolium-5-Carboxanilide [XTT]) reduction assay. To assess the activity on AgNPs against C. auris preformed biofilms, cells were first added to the wells of the microtiter plates and incubated at 37° C. for 24 h to allow for biofilm formation. The plates were then gently washed, and serial dilutions of AgNPs at concentrations ranging from 2.3 to 0.017 ppm added. The plates were sealed with parafilm and incubated for an additional 24 h. Then, the plates were carefully washed, and the biofilms quantified using the XTT reduction assay. All tests were performed in duplicate in independent experiments and were repeated at least three times. The data from the dose-response curves was used to calculate IC₅₀ values using a four-parameter Hill equation using SigmaPlot software (version 10.0, Systat Software, Inc., San Jose, Calif.).

Prevention of C. auris Biofilm Formation by Functionalized SESs with AgNPs

For C. auris inhibition of biofilm formation inhibition on SESs a protocol was adapted to examine the inhibitory effect of Ag NPs against C. auris biofilm formation on the surface of a functionalized catheter substrate. Briefly, silicone elastomer sheets (Bentec Medical, Woodland, Calif., USA) were cut with scissors into 1 cm² pieces, washed with laboratory detergent, rinsed with distilled water immediately, and sterilized by steam autoclave. The sterile SESs were pre-treated with fetal bovine serum (FBS) at 37° C. for 24 h. After washing in sterile PBS twice to remove the excess FBS, SESs were placed on the bottom of a sterile 24-well plate (Corning). To functionalize the SESs with AgNPs, 2 ml of RPMI medium containing either AgNPs at concentrations ranging from 2.3 to 0.07 ppm, or no AgNPs (control) was added to individual wells containing the SESs, which were then incubated at 37° C. for 24 h. The SESs were then washed thoroughly three times with sterile PBS to remove non-attached AgNPs. Energy Dispersive X-Ray Spectroscopy (EDS) spectra of AgNPs was used on the surface of SES to demonstrate the presence of Ag signal in the sample by spectral mapping acquisition. The Energy Dispersive X-ray (EDX) microanalysis (Hitachi S-5500 SEM) due to its high sensitivity in detecting the different nanoparticles on surfaces was also used to detect AgNPs placed on the SESs.

After functionalizing the SESs with different concentrations of AgNPs, in order to measure biofilm formation inhibition on the functionalized SESs fresh RPMI media containing 2 ml of a 5×10⁶ cells/ml suspension of C. auris were added and the 24 well-plates incubated in an orbital shaker at 100 r.p.m. for 2 h at 37° C. After this adhesion step, the SESs were washed three times with sterile PBS to remove non-attached yeast cells. Fresh RPMI media was added to the wells containing the SESs and the plates were incubated in an orbital shaker at 37° C. (100 r.p.m) for 24 h. After incubation this the SESs were washed three times with sterile PBS and processed using the XTT-reduction assay, to calculate the extent of biofilm inhibition compared to the untreated SES. All tests were performed in duplicate in independent experiments and were repeated at least three times.

Prevention of C. auris Biofilm Formation by Functionalized Bandage Dressings with AgNPs

To test the inhibition of C. auris by textiles loaded with AgNPs, the fabrics (elastic bandage wraps, EWBs) were cut into 1 cm² pieces, washed with detergent, rinsed with distilled water immediately, and sterilized by steam autoclave. EBWs were placed in wells of 24-well microtiter plates, and the wells loaded with AgNPs from 2.3 to 0.002 ppm dissolved in sterile RPMI (500 μL per well). After 24 h, all the functionalized EWBs including controls were thoroughly washed with sterile PBS for 3 times to eliminate the unattached AgNPs. To document the attachment of the AgNPs on the EBWs samples were observed by SEM. For biofilm formation, 2 mL of C. auris (5×10⁶ yeast cells/mL in RPMI medium) were added to each well containing the silver-loaded EWBs, and the plates incubated at 37° C. for 2 h in an orbital shaker at 100 r.p.m. The nonadherent cells were removed by gentle washing two times with sterile PBS, and the silver-loaded EBWs were transferred to the wells of a new 24 well-plate and incubated 24 h at 37° C. After washings, the metabolic activity of yeast cells was measured by the XTT reduction assay to calculate the percent of inhibition C. auris growth. All tests were performed in duplicate in independent experiments and were repeated at least three times.

Antifungal Protection of Functionalized Bandage Dressings after Multiple Successive Washes

Following the method described above, functionalized silver-loaded EBWs were washed tree times with PBS every 24 h. After washing at the indicated day (0 to 8 day), the washed silver loaded dressings were tested for their ability to inhibit C. auris biofilm formation as compared to positive (untreated EBWs with C. auris) and negative controls (untreated EBWs without AgNPs) in order to assess the protection of the silver-loaded dressings against C. auris after each washing cycle every 24 h. The percent inhibition of C. auris biofilm formation was estimated by the XTT reduction assay as indicated above. All tests were performed in duplicate in independent experiments and were repeated at least three times.

Scanning Electron Microscopy (SEM) of C. auris Biofilms on Different Surfaces.

For SEM, C. auris biofilms were cultured on 48-well polystyrene tissue culture plates (Corning) at 37° C. for 24 h for high resolution SEM ultrastructural observation. The preformed biofilms were then treated with or without AgNPs (0.48 ppm) for an additional 24 h. After treatment, the biofilms were gently washed three times in sterile PBS, and fixed with 4% formaldehyde and 1% glutaraldehyde at room temperature for 1 h. The fixed samples were washed three times in PBS and then stained for 1 h at RT in 1% osmium tetroxide (OsO₄). After washing the biofilms with PBS, samples were dehydrated through a series of ethanol concentrations (25%, 50%, 70%, 95% (10 min each), and absolute alcohol (for 20 min). The stained dehydrated biofilms were then analyzed by SEM in a Hitachi S-5500 (Hitachi Ltd., Tokyo, Japan).

For C. auris inhibition of biofilm formation inhibition on functionalized SESs, and to document the functionalization of the EBWs, and the ultrastructural effect of the silver-loaded EBWs or EBWS alone on the inhibition of C. auris, samples prepared as explained above were fixed with 4% formaldehyde and 1% glutaraldehyde at room temperature for 1 h. Samples were then visualized using variable-pressure high-resolution scanning electron microscopy (VP-SEM, SU1510, Hitachi, Tokyo, Japan) at 20 kV, under low-vacuum mode.

Activity of AgNPs Against C. auris Biofilms.

Candidiasis represents one of the most frequent nosocomial infections and the most common invasive fungal opportunistic infection worldwide. In particular, intensive care unit (ICU) acquired Candida BSIs carry high mortality rates. Although a majority of infections are caused by C. albicans, a recent shift towards non-albicans Candida (NAC) species with increased resistance to antifungals, and most recently C. auris has emerged as a formidable opportunistic pathogen capable of causing major outbreaks in health care facilities. C. auris is capable of forming biofilms which are associated with virulence and poorer outcomes for patients. These C. auris biofilms are instrinsically resistant to all classes of clinically-used antifungals, this resistance was also previously reported for this C. auris 0390 strain, and biofilm formation may also contribute to the persistence of C. auris on environmental surfaces by allowing survival for extended periods of time on dry or wet surfaces. Thus, there is an urgent need for the development of novel approaches to control C. auris infections, in particular those associated with biofilm formation. With microwave formed AgNPs, energy transfer is faster and with better uniformity to produce nanoparticles in large scale. This methodology produces substantially pure metallic nanoparticles without added reducing agents that could be toxic or contaminants to the environment, which is ideal for biomedical applications. Thus, after initial characterization of the nanoparticles to confirm their proper synthesis, the activity of this type of nanoparticles against C. auris biofilms was evaluated.

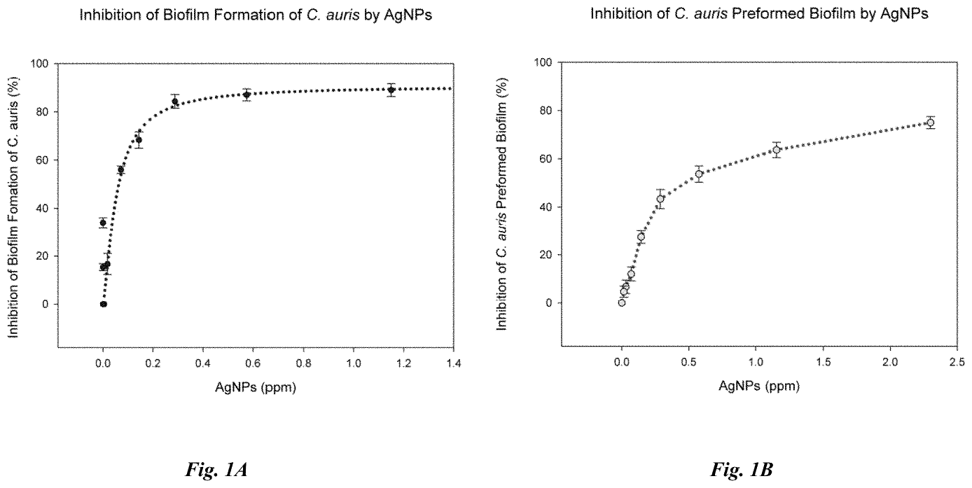

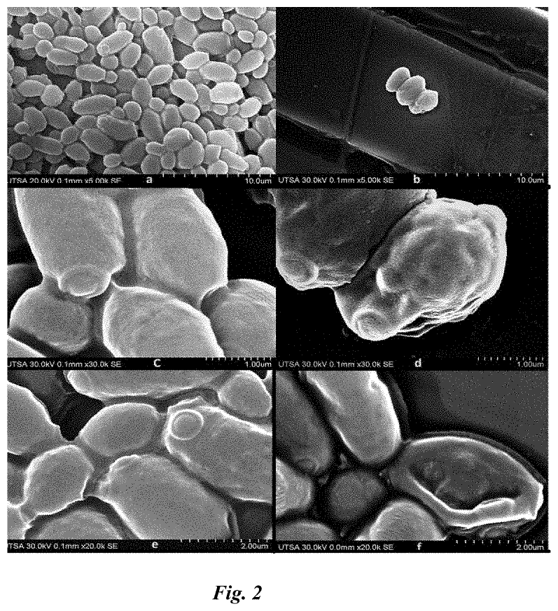

In a first set of experiments a 96-well microtiter plate model was used to assess the anti-biofilm activity of AgNPs against C. auris. More specifically, the ability of AgNPs to inhibit C. auris biofilm formation was evaluated, as well as their activity against C. auris preformed biofilms. Results indicated a potent inhibitory effect of AgNPs, in a dose-dependent manner, on C. auris 0390 strain biofilm formation, with a calculated IC₅₀ of 0.06 ppm (FIG. 1A). AgNPs also demonstrated efficacy when tested against pre-formed biofilms of the same C. auris strain, although at higher concentrations than those required to inhibit biofilm formation, resulting in a calculated IC₅₀ of 0.48 ppm (FIG. 1B).

SEM advanced electron microscopy (AEM) was used to directly visualize the ultrastructural effect of AgNPs against preformed C. auris biofilms (after 24 h incubation) at a concentration of 0.48 ppm, which was determined to be the IC₅₀ dose (FIG. 2). The preformed biofilm (control without AgNPs) showed a characteristic dense network of agglomerated and well-defined C. auris yeast cells with smooth cell surface, oval shaped yeast morphology, with several budding cells and plenty of EPS surrounding the fungal cells (FIG. 2, panels a, c, and e). The ultrastructural analysis obtained under SEM techniques after treatment with AgNPs of the C. auris biofilm showed scarce cells as observed in FIG. 2, panel b with major changes in the shape and surface appearance of the yeasts from smooth to rough cell surface, indicating CW damage, disruption and distortion of the outer surface of the yeast wall (FIG. 2, panels b, d and f).

Inhibition of C. auris Biofilms on SESs Functionalized with AgNPs.

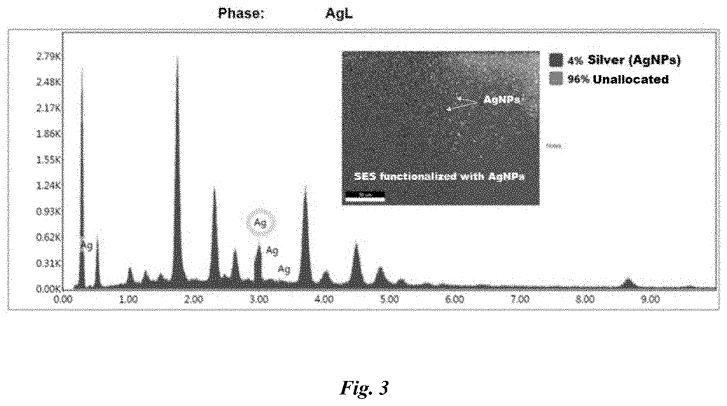

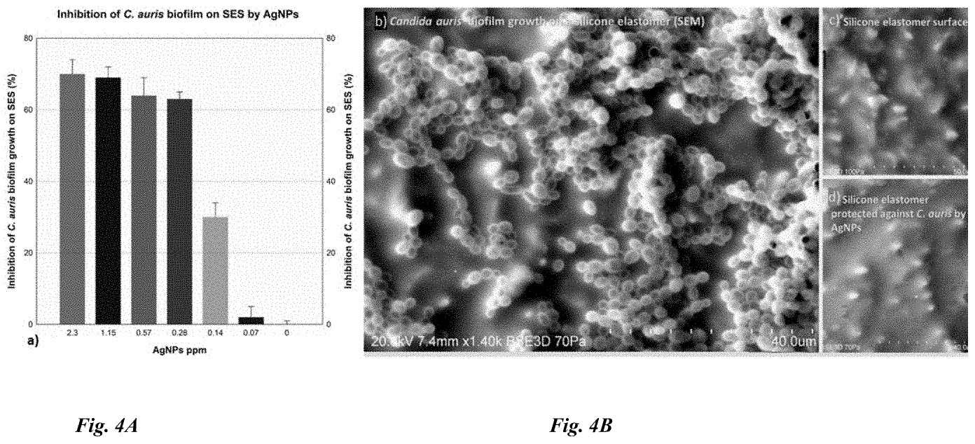

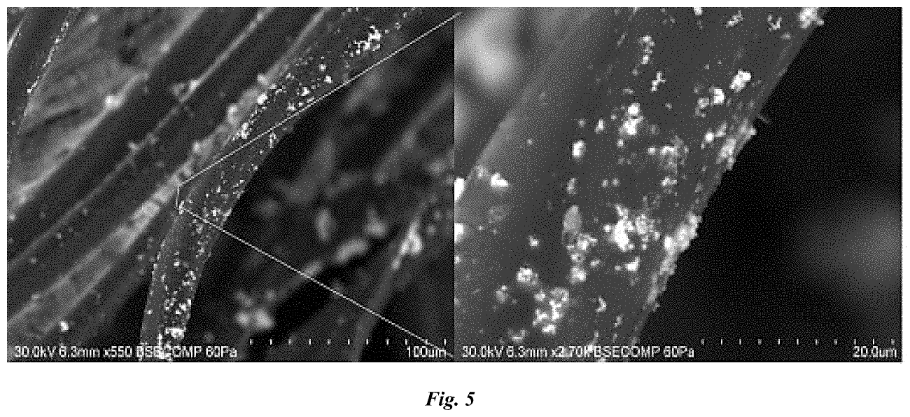

C. auris causes catheter-related fungemia associated with high mortality. This is often the result of biofilms formed in catheterized patients, which as mentioned before are intrinsically resistant to all antifungals in clinical use for the treatment of invasive candidiasis. An attractive alternative is to prevent colonization and biofilm formation by coating biomaterials with biofilm inhibitors. Therefore, after having established the anti-biofilm activity of AgNPs against C. auris, functionalizing the surface of catheters with AgNPs may lead to the inhibition of C. auris biofilm formation was investigated by using a modified assay in which different concentrations of AgNPs were incubated with SESs to directly coat the substrate of catheters before the addition of C. auris cells for biofilm formation. Prior to biofilm inhibition experiments SEM and EDS was used to confirm the effective functionalization of the elastomers' surface with AgNPs (FIG. 3). EDS demonstrated the presence of Ag signal in the sample by spectral mapping acquisition, detecting AgNPs (red dots) placed on the SESs, with optical absorption band peaking at 3 keV confirming the presence of metallic silver on the surface (FIG. 3). As seen in FIGS. 4A and 4B, functionalization of the SES with AgNPs resulted in significant inhibition of biofilm formation in a dose response manner as compared to the untreated control, with concentrations of AgNPs from 2.3 to 0.28 ppm leading to more than 50% inhibition of C. auris biofilm formation (FIG. 4A).

These results were confirmed by advanced electron microscopy analysis by SEM. SESs with C. auris biofilm (control without AgNPs) formed onto the flat, rough surface of the silicone elastomer showed abundant cells growing in agglomerates, yeasts cells appear with characteristic oval-shaped morphology as well as several budding yeasts (FIG. 4B, panel B). Clean SES is observed in the negative control (without AgNPs and without C. auris) with the characteristic roughness appearance on the surface of the elastomer (FIG. 4B, panel c). In contrast, no biofilm formation was observed on SES functionalized with AgNPs applied onto the elastomer surface (FIG. 4B, panel d).

Inhibition of C. auris Biofilms on EBWs Functionalized with AgNPs.

One of the major factors contributing to the emergence and fast spread of C. auris as an opportunistic pathogen, and to becoming the causative agent of major outbreaks in health care facilities, is its ability to persist on different types of environmental surfaces. It is likely that biofilm formation is associated with C. auris persistence and growth on these environmental surfaces leading to its protection from disinfectants. However, current data on the efficacy of products and methods for the disinfection of C. auris—contaminated environmental surfaces is scarce, as highlighted in a recent review on this topic. Thus, it is also possible that AgNPs may also be used in the control and disinfection of contaminated surfaces. To this extent, and as an initial approach, the ability of EBWs dressings loaded with AgNPs to inhibit C. auris biofilm formation, as representative of a typical hospital environment including fabrics and other products commonly found in a health care setting (i.e. dressings, bed linens, patient clothing, medical gowns and garments, etc.) was investigated. For these experiments EBWs were functionalized with different concentrations of AgNPs, ranging from 2.3 to 0.002 ppm. Upon incubation, AgNPs (2-3 nm diameter) attached to the dressing fibers and were clearly visible in the SEM images after three thorough washes with PBS (FIG. 5).

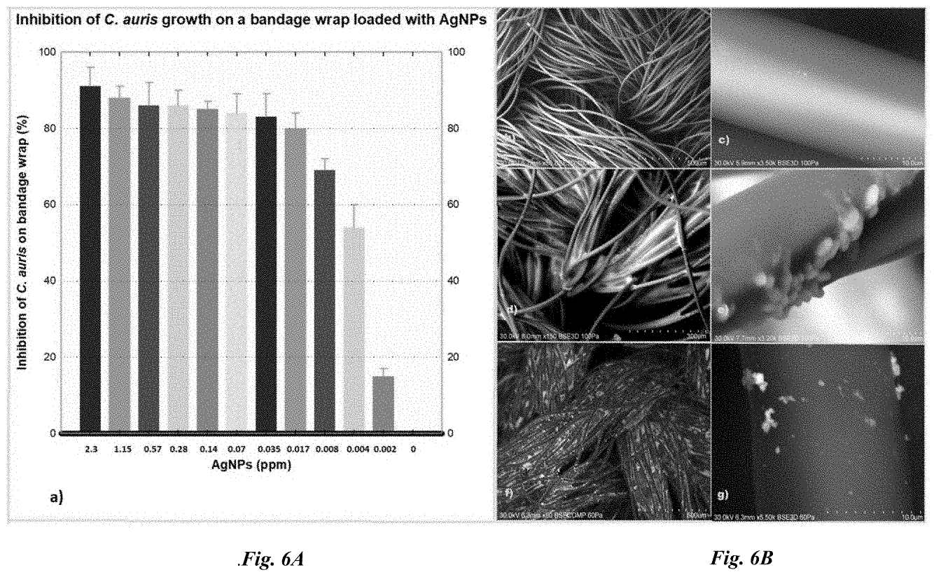

Results indicated that an inhibition of more than 80% in C. auris biofilm formation was achieved in dressings loaded with AgNPs at concentrations from 2.3 to 0.017 ppm (FIG. 6A). Dressings loaded with lower concentrations of AgNPs (0.008 to 0.002 ppm) still inhibited C. auris growth by more than 50%. These results show that effective inhibition of C. auris growth on EBWs dressings can be achieved at relatively low concentrations of AgNPs when loaded on EBWs. The effect of functionalizing EBWs with AgNPs was also examined by advanced electron microscopy (FIG. 6B, panels b-g). FIG. 6B, panels b, c show the smooth surface observed for negative control EBWs (without nanoparticles and without C. auris). In the absence of nanoparticles, growth of C. auris attached to the fibers was clearly visible in the SEM images, the extracellular matrix of the resulting biofilm acting as a glue between the fibers was noticeable at lower magnification (FIG. 6B, panel d), whereas at higher magnification aggregates of C. auris cells were observed on the surface of the fibers (FIG. 6B, panel e). In stark contrast, no biofilm growth was visible on functionalized fibers with AgNPs (FIG. 6B, panels f, g).

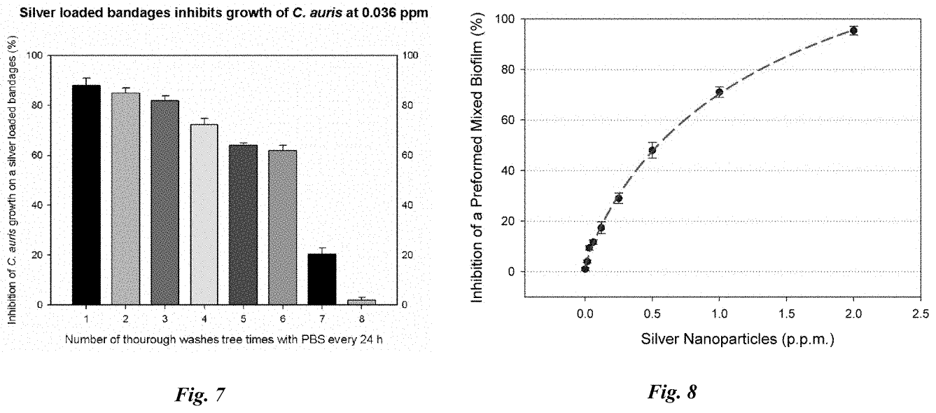

In follow up experiments, dressings previously loaded with AgNPs (0.036 ppm) were washed thoroughly three times with PBS every 24 h for 8 consecutive days, and evaluated for their ability to still inhibit C. auris biofilm formation. As shown in FIG. 7, results indicated that effective protection against the C. auris growth was achieved from cycles 1 to 3 (over 80% inhibition), and from cycles 4 to 6 (over 50% inhibition). On day 7 the protection was much diminished (less than 20% inhibition). Thus, functionalized EBWs with AgNPs retained their antifungal activity against C. auris after multiple washes with PBS, indicating the sustained inhibition of C. auris growth on functionalized EBWs dressings over longer periods of time and at relatively low concentrations of AgNPs.

Overall, the potent inhibitory activity of AgNPs on both medical and environmental surfaces against C. auris biofilms point to a potential role for AgNPs in the prevention, treatment and control of these devastating infections, which are all urgently needed to curtail the spread of this emerging pathogen. It is believed that this is the first study showing potent in vitro activity of AgNPs against C. auris biofilms on different biomedical applications.

### Inhibition of Combined Fungal and Bacterial Biofilm Formation

Due to the intrinsic recalcitrance of mixed fungal/bacterial biofilms against conventional antibiotic treatment the in vitro activity of AgNPs against these cross-kingdom biofilms was examined. Mixed C. albicans/MRSA biofilms were grown on the bottom of wells of 96-well microtiter plates, and the preformed mixed biofilms were exposed to a range of concentrations of AgNPs. Results demonstrated a potent dose-response activity against these fungal/bacterial biofilms, with a calculated IC₅₀ value of 0.53 ppm or 530 ng/mL (FIG. 8).

### Ultrastructural Effects of AgNPs on Mixed Biofilms Using Scanning Electron Microscopy (SEM)

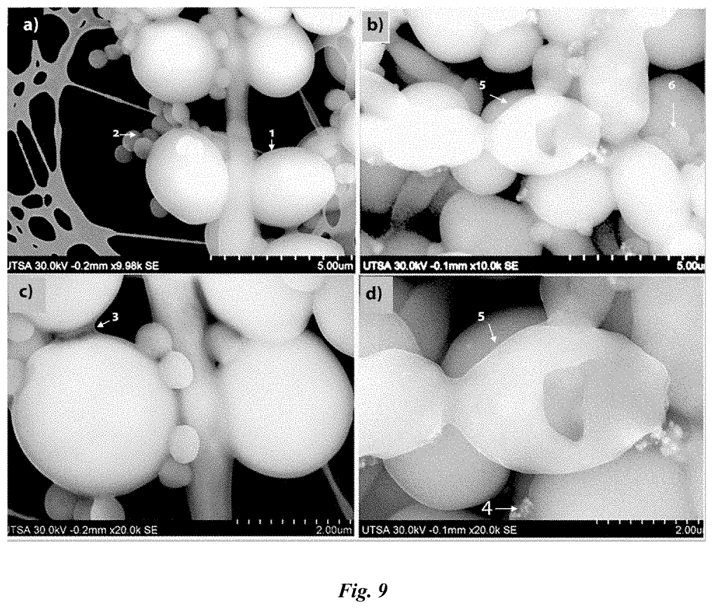

An advanced SEM technique was performed to report the ultrastructural effects of AgNPs against mixed biofilms of C. albicans and MRSA (FIG. 9). The preformed biofilm without AgNPs as shown in FIG. 9, panels a and c show characteristic MRSA cocci cells attached to the fungal elements, both yeast and hyphal cells, with C. albicans cells displaying a smooth cell surface. The biofilm exopolymeric substances were also clearly visible in FIG. 9, panels a and c. This was in stark contrast to SEM images obtained after 24 h treatment of the mixed fungal/bacterial biofilms with AgNPs, at a concentration of 0.53 ppm, demonstrating changes in both the shape and surface appearance of the fungal cells, indicative of cell wall/surface disruption, and with many fewer bacterial cells attached to the fungal elements as shown in FIG. 9, panels b and d.

Inhibition of Mixed Biofilm on the Surface of the Functionalized Elastomer with AgNPs

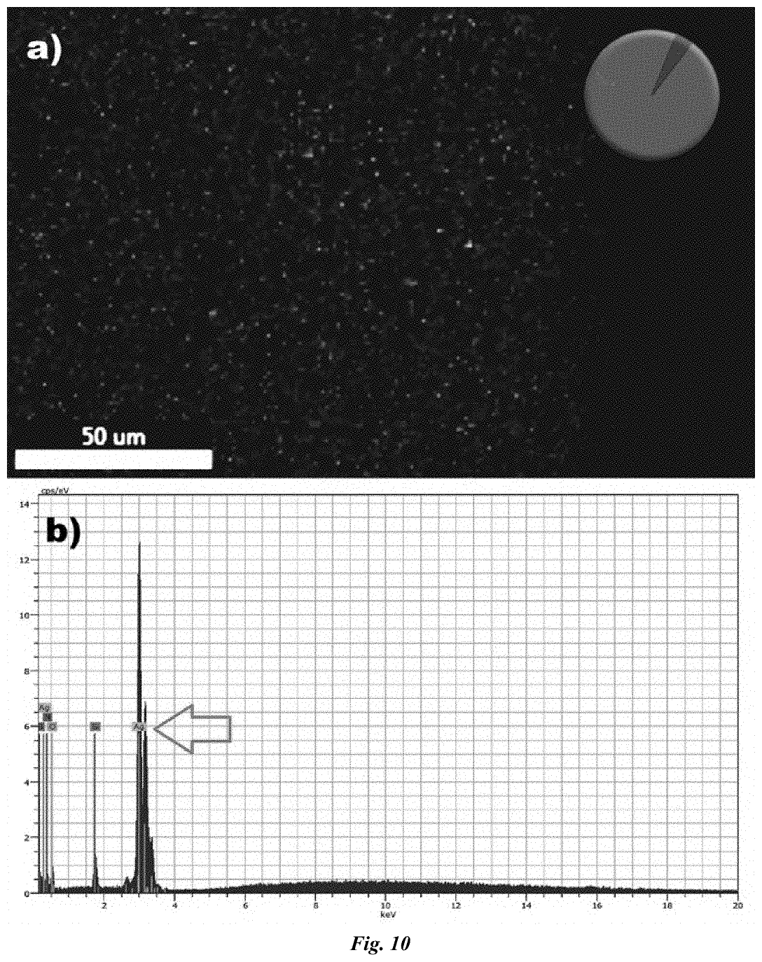

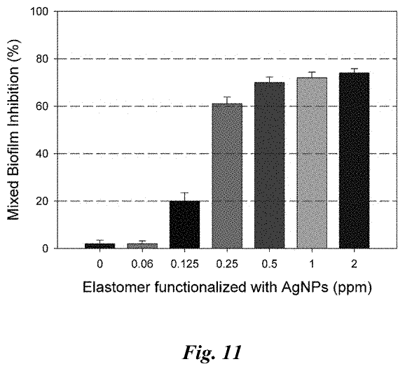

Mixed biofilms formed by these two opportunistic pathogens often cause catheter-related blood stream infections associated with high mortality. Thus, after having established the potent activity of AgNPs against dual-species biofilms of C. albicans and S. aureus, functionalization of catheter materials with nonantibiotics such as AgNPs was investigated to determine whether AgNPs could provide for an effective strategy to prevent mixed biofilm formation. In a first set of experiments medical grade silicone elastomers was functionalized with substantially pure AgNPs. The functionalization of the silicone elastomers with AgNPs (0.53 ppm) or without AgNPs (control) after thorough washings was demonstrated by the presence of AgNPs on the surface of the elastomers by using highly sensitive Energy Dispersive X-ray (EDX) microanalysis. Functionalized Silicone elastomers were scanned by spectral mapping and the red dots in FIG. 10 panel a show the signal of AgNPs demonstrating the presence of the signal of the element.

Once the effective functionalization was demonstrated, further functionalization of catheter surfaces with positively charged AgNPs was tested to determine whether mixed biofilm formation would be inhibited. A modified assay in which functionalized silicone elastomers with different concentrations of substantially pure positively charged AgNPs were used as the substrates for biofilm formation. Results indicated that, as compared to the untreated control, functionalization of the elastomer with a range of concentrations of AgNPs (from 0.06 to 2.0 ppm) effectively inhibited the formation of mixed C. albicans/MRSA biofilm. FIG. 11 shows the extent of mixed biofilm inhibition growth on silicone elastomers functionalized with different concentrations of AgNPs. The dose response inhibition was demonstrated by a resazurin reduction assay (% viability) on functionalized elastomers at different concentrations of AgNPs (0 to 2 ppm), showing the inhibition of the mixed biofilms in a dose response manner. Error bars represent standard deviation (SD) of the means.

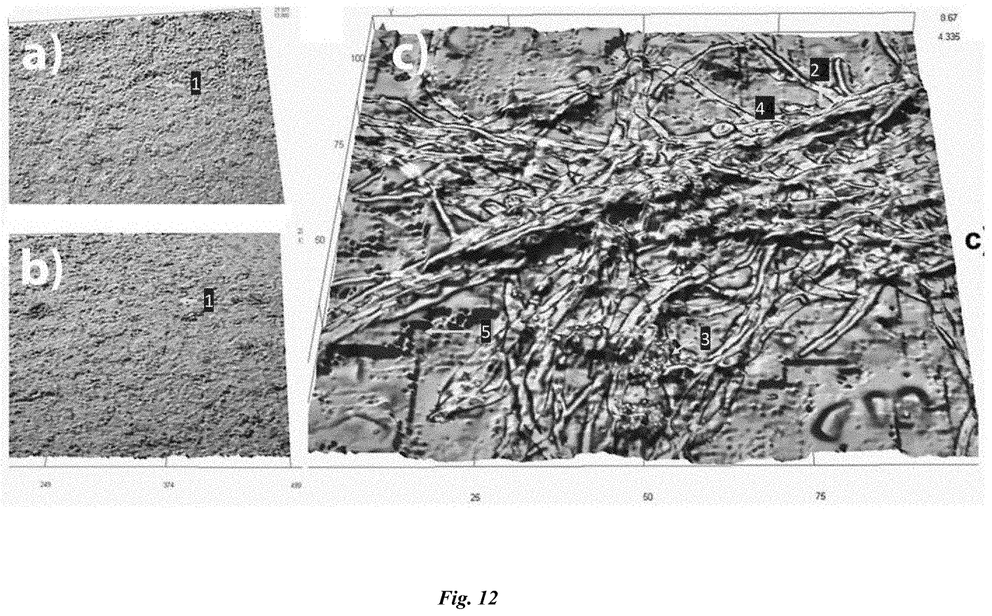

High-resolution opto-digital microscopy was then used to further document the inhibitory effect of functionalized silicone elastomer on mixed biofilm formation. After analyzing the effective dose response inhibition of growth of the functionalized silicone elastomers by a viability assay (FIG. 11) and to document high-resolution images in 2D of the growth inhibition of the mixed biofilm, the functionalized (AgNPs 0.53 ppm) and non-functionalized silicone elastomers were observed with an opto-digital microscope, as shown in FIG. 12. As shown in FIG. 12, panel a, functionalization of the same silicone elastomers with AgNPs (0.53 ppm) effectively prevented mixed biofilm formation. In contrast mixed C. albicans/MRSA biofilm were able to grow on the surface of the control un-functionalized silicone elastomers, with abundant presence of fungal hyphal elements and MRSA colonies embedded within the extracellular matrix as shown in FIG. 12, panel c.

Biofilms are consortia of microbial cells attached to a substrate and embedded within a matrix of self-produced exopolymeric materials. Both bacteria and fungi are capable of forming biofilms, and a majority of infections are associated with a biofilm aetiology. By virtue of their characteristics, cells within these biofilms are protected against host immune mechanisms and also display high levels of resistance against most antibiotics. Mixed fungal/bacterial biofilm infections are particularly hard to treat. Together, C. albicans and S. aureus are responsible for a majority of opportunistic nosocomial infections, and they are often co-isolated from a host. Frequently these polymicrobial infections are associated with the formation of mixed biofilms in catheters and other indwelling devices, where C. albicans and S. aureus display a symbiotic relationship. For example, MRSA resistance is enhanced within the mixed biofilm due to protection by the fungal extracellular matrix, more specifically the secreted β-1,3-glucan component, and the invasive behavior of MRSA is facilitated by C. albicans leading to invasive infection in co-colonized patients. The ultimate effect is increased mortality and morbidity rates, with significant costs to the health care system.

Because cells within polymicrobial biofilms exhibit high levels of resistance to antibiotic treatment, alternative approaches are urgently needed to combat the threat of these biofilm-associated infections. Substantially pure, positively charged AgNPs activity against dual-species C. albicans/S. aureus biofilms was, therefore, tested. The results demonstrated a potent dose-response activity of AgNPs against preformed mixed C. albicans/MRSA biofilms.

Catheter-related bloodstream infections are the cause of approximately one-third of all healthcare-acquired infection deaths. The use of indwelling medical devices in hospitalized patients offers favorable conditions for microbial biofilm growth, and most often these two opportunistic pathogens (C. albicans and S. aureus) interact with each other and form mixed biofilms within this setting. Antimicrobial-coated catheters have been proposed to decrease the chances to acquire a CRBSI, and an attractive alternative is to prevent colonization and biofilm formation by coating biomaterials with biofilm inhibitors. The ideal antimicrobial catheter should offer a low-cost application technology, long-term broad-spectrum antimicrobial surface effect, and without side effects or toxicity. Nanotechnology-based approaches are designed to control and eradicate catheter-related bloodstream infections. Therefore, after having established the potent activity of the AgNPs against these mixed fungal/bacterial biofilms in the standard 96-well microtiter plate model, the ability of AgNPs to inhibit mixed biofilm formation was tested within a clinically-relevant model, more specifically when used to functionalize the surface of silicone elastomers. Results from this set of experiments clearly indicated that functionalization of the elastomer with AgNPs resulted in significant inhibition of biofilm formation in a dose-response manner as compared to the untreated control. These results were further verified by using opto-digital microscopy. An opto-digital microscope incorporates conventional optical microscope, digital multimedia acquisition, and digital processing software to obtain highest quality images allowing to display the image details. The resulting images corroborated the almost complete lack of biofilm formation in silicone sheets functionalized with AgNPs, as compared to control, non-functionalized, elastomers.

Overall the results confirm the efficacy of AgNPs against mixed biofilms of C. albicans and S. aureus, and add to a growing body of evidence pointing to the activity of different types of nanoparticles against a variety of pathogenic microorganisms, including those capable of forming biofilms which are typically recalcitrant to clinically-used antibiotics and for which there is an urgent need to develop preventive and therapeutic alternatives. This is where novel nanotechnological approaches, alone or in combination with conventional antibiotic therapy, may play an important role. Although the results indicate a strong potential of silver nanoparticles for the prevention and treatment of highly resistant polymicrobial biofilm-associated infections. Due to the complex interactions of silver with living tissues, important considerations regarding their biocompatibility and cytotoxicity need to be taken into account for their eventual pre-clinical and clinical development to combat the threat of mixed biofilm infections.

### Microbial Strains, Media and Culture Conditions

To culture mixed preformed biofilms in this study, the fungus C. albicans (SC5314), and the methicillin-resistant strain of S. aureus (MRSA TCH1516) were used. For long-term cryopreservation of C. albicans stocks were stored in 15% glycerol into an ultrafreezer (−80° C.), to maintain the yeast strain, yeast peptone dextrose (1% yeast extract, 2% peptone, 2% dextrose, YPD) agar plates were used and kept at 4° C. Single yeast colonies were transferred from YPD plates into 10 ml of YPD liquid media for culturing C. albicans, which was routinely grown in an orbital shaker (180 rpm) at 30° C. overnight. Cells were pelleted by centrifugation at 5000×g for 5 min and the supernatant was decanted, then the resuspended cell pellet was washed twice in sterile phosphate-buffered saline (PBS, consisting of 137 mM NaCl, 2.7 mM KCl, 10 mM Na₂HPO₄ and 2 mM KH₂PO₄; pH 7.2) followed by a vortexing step of 2 min and centrifugation, dilutions (100-fold) of the suspended cells were prepared for biofilm growth and counted using a hemocytometer on a bright field microscope. Yeast cells were resuspended at a final concentration of 1.0×10⁶ cells/mL in the corresponding medium, to be seeded for biofilm formation in the 96-well microtiter plates (see below). Stock cultures of S. aureus MRSA TCH1516 were cryopreserved in aliquots at −80° C. in Brain Heart Infusion (BHI) broth (Difco, Becton Dickinson, Sparks, Md., USA) with 50% glycerol for long-term storage. A sterile applicator stick was used to streak out a small amount of inoculum from the frozen stock onto a selective chromogenic plate (BBL CHROMagar, BD Diagnostics, HD, Germany) and stored at 4° C. Prior to each experiment plates were incubated for 16-24 h at 37° C., then a loopful of each stock culture was inoculated into 10 mL of Tryptic Soy Broth (TSB) liquid media at 37° C. for 24 h. The bacterial culture was sedimented by centrifugation (3600×g for 10 min at 4° C.), washed and resuspended in PBS and used for counting. The bacterial count of the inoculum was determined and resuspended to the final concentration (1×10⁷ CFU/mL) on BHI broth supplemented with 10% human serum on 96 well plates and incubated at 37° C. for 18 h. TSB and BHI have been previously determined to be optimal media for supporting both C. albicans/Staphylococcus aureus (dual species) biofilm.

### Preparation and Characterization of Substantially Pure AgNPs

AgNPs were obtained by a physical method (microwave irradiation-assisted synthesis) using the Ethos EZ microwave, a high-performance microwave digestion system (Milestone Inc., Shelton, Conn., USA) as described above, resulting in the production of substantially pure, round silver nanoparticles. This technique allows a fast rise in initial temperatures in the heat reaction. Briefly, 1.7 g of AgNO₃ was dissolved in 10 mL of distilled water (DI) and treated by MW irradiation. The AgNO₃ solution was continuously irradiated for 15 s at 1000 W. After MW irradiation, samples were cooled to room temperature (RT). The Transmission Electron Microscope (TEM) analysis with high-resolution images (JEM-2010, Jeol Ltd., Tokyo, Japan) was used to measure the average particle size distribution and shape of the AgNPs. AgNPs were in average 1-3 nm and rounded in shape (not shown). The physicochemical characterization of the substantially pure AgNPs was performed. Briefly, the concentration of the solution after MW-irradiation was measured in part per million (ppm) by an atomic absorption spectrophotometer (AA-6200, Shimadzu Corporation, Kyoto, Japan); this technique is precise and sensitive therefore is the most used method in analytical measures for the metal concentration in a solution. To demonstrate the surface charge of the nanoparticles a Zetasizer Nano ZS (Malvern Instruments Ltd, Malvern, Worcestershire, UK) in solution at 25° C. was used. Over a time period of 120 h the Zeta potential (ZP) shifts to a positive surface charge indicating that this AgNPs become positively charged.

### Formation of Mixed Fungal/Bacterial Biofilms in 96-Well Microtiter Plates

One hundred μl of the prepared dilutions with mixed microorganisms (1×10⁶ cells/ml for C. albicans, 1×10⁷ cells/ml for MRSA) in 1:1 v/v YPD/BHI added with 10% human serum were pipetted into each well of a sterile 96-well polystyrene tissue culture plates (Corning® Incorporated, Corning, N.Y., USA). The plates were then incubated for 24 h at 37° C. After incubation, the supernatant from each well was decanted and planktonic cells were removed by washing with 100 μl PBS. The viability of cells within the biofilms was estimated by adding 100 μl of 1:10 v/v Presto Blue Cell Viability Reagent (Invitrogen, Carlsbad, Calif., USA) in 1:1 v/v YPD/BHI media and incubated for 30 min at 37° C. Finally, 80 μl from each well were transferred into a new 96-well plate for fluorescent readings. The microtiter plate reader (BioTek® Synergy HT, Winooski, Vt., USA) was set to measure fluorescence at 530/25 nm excitation and 590/35 emission.

### In Vitro Activity of AgNPs Against Mixed Fungal/Bacterial Biofilms

AgNPs susceptibility testing was performed by adding AgNPs at two-fold serial dilutions concentrations to preformed mixed species biofilms grown in 1:1 v/v YPD/BHI+10% human serum, which were then incubated for an additional 24 h in the presence of AgNPs. Briefly, wells of microtiter plates were seeded with mixed microorganisms as described above and incubated for 24 h to allow for biofilm formation. AgNPs were diluted in RPMI and added to the preformed biofilms (after tree PBS washings) at the following final concentrations: 2.0 to 0.015 ppm in YPD/BHI plus serum media, or without the AgNPs as the non-treated control and the medium alone as the blank control. After incubation for an additional 24 h, microtiter plates were washed and processed using the Presto Blue assay as described above. Additionally, IC₅₀ values for AgNPs were determined by SigmaPlot® plot analysis, using the four-parameter logistic nonlinear regression equation. All assays were performed in duplicate in independent experiments and were repeated at least three times.

### Pretreatment, Functionalization and Characterization of Medical Grade Silicone Elastomers

Medical grade silicone elastomer sheets were cut (1 cm²), washed with medical grade detergent, then wash off all detergent with several changes of distilled water and disinfected by steam sterilization (autoclave). The rubber sheets were treated overnight at 37° C. with sterile fetal bovine serum (FBS). Then elastomers were washed twice to rinse off the FBS and were placed into sterile 48-well culture plate. Silicone elastomers were functionalized with AgNPs, as described above. RPMI medium (2 mL) with either AgNPs at different concentrations (0.02 to 2 ppm) or without AgNPs (control) in a sterile 48-well microtiter plates was then added to the functionalized silicone elastomers where then incubated overnight at 37° C. The pieces were washed three times with sterile phosphate buffered saline buffer to remove unattached nanoparticles. To confirm the presence of AgNPs attached on the silicone elastomers spectral mapping acquisition by scanning electron microscopy/energy dispersive X-ray spectrometry (SEM/EDS) (Hitachi S-5500 SEM). This technique of elemental analysis is based on the generation of characteristic X-rays, is energy-specific to the silver atoms of the specimen by the incident beam of electrons. EDX microanalysis is used to qualitatively map whether elements in the spectrum are present at specific sites.

### Inhibition of the Mixed Biofilms on the Surface of the Functionalized Elastomer by AgNPs

Silicone elastomers functionalized with AgNPs (2 to 0.02 ppm) and non-functionalized elastomers were tested to ensure the inhibition of mixed biofilm growth. Briefly, RPMI media (1 mL) with 5×10⁶ cells/mL of C. albicans cocultured with 1 mL of 5×10⁷ MRSA in MOPS-buffered RPMI 1640 (pH 7.0), and placed in sterile 24-well culture plates, incubated in an orbital shaker (100 rpm) at 37° C. After 2 h (adhesion step), the rubber sheets were washed twice with 2 mL of PBS at room temperature to remove detached (planktonic) cells. Culture plate containing the elastomers were placed in an orbital rotatory shaker at 37° C. and 100 rpm overnight. The rubber sheets were washed thrice (PBS). The viability of cells was measured by Presto Blue Cell Viability Reagent as mentioned above, to calculate the biofilm inhibition in functionalized sheets as compared to the nonfunctionalized elastomer (control). All silicone elastomers were observed under opto-digital microscopy to corroborate the results (see below). All assays were performed in duplicate in independent experiments and were repeated at least three times.

### SEM Assessments

Mixed biofilms were cultured at 37° C. for 24 h, as described above for observation in high resolution SEM. Briefly; on 48-well polystyrene tissue culture plates, mixed preformed biofilms were then treated with or without AgNPs (0.53 ppm) for another 24 h at 37° C. After treatment, the attached biofilm was washed three times in sterile saline (PBS) and fixed with 4% formaldehyde (FA) and 1% glutaraldehyde (GA) at room temperature (RT) for 1 h. The fixed samples were gently washed three times in PBS and then post-fixed for 1 h at RT in 1% osmium tetroxide (OsO₄) in a fume hood and then dehydrated through a series of ethanol concentrations (25%, 50%, 70%, 95% (10 min each), and absolute alcohol (for 20 min). The stained dehydrated mixed biofilm was then mounted on a 300-mesh carbon-coated copper grids were observed by SEM in a Hitachi S-5500 (Hitachi Ltd., Tokyo, Japan).

### Opto-Digital Microscopy of the Mixed Biofilm on Silicone Elastomers

Visualization of biofilms formed on silicone elastomers using opto-digital visualization 2D was used to document the biofilm-inhibitory effect of catheter materials functionalized by AgNPs, an opto-digital microscope (DSX 500, Olympus Corporation, Japan) was employed. Silicone elastomer sheets were pretreated and functionalized as indicated above. To ensure uniform biofilm formation on the functionalized or non-functionalized silicone elastomer sheets, 1 ml of a 5×10⁶ yeast cells/mL suspension of C. albicans was cocultured with 1 mL of 5×10′ MRSA in MOPS-buffered RPMI 1640 (pH 7.0), added onto the elastomers and incubated in an orbital shaker (New Brunswick Scientific, Edison, N.J., USA) at 100 rpm. After for 2 h incubation at 37° C., the nonadherent cells were removed by gentle washing two times with sterile PBS, then elastomers were placed on sterile wells in a 24-well plate. After incubation for 24 h at 37° C., elastomers were washed twice with sterile PBS and fixed with 4% formaldehyde (FA) and 1% glutaraldehyde (GA). After 1 h fixation at room temperature (RT) elastomers were observed on the surface to document the morphology of the mixed biofilm attached to the surface of the sheets by DSX500 High-resolution opto-digital microscope (ODM) to image and to visualize the biofilm growth or inhibition on the functionalized elastomers. ODM captured 2D images of the surface of the elastomers as ODM is a reliable method for biomedical exploration purposes.

Inhibition of Viral Transmission Trough Textile with or without AgNPs Functionalization Using a Transwell.

Functionalized textile (0.28 ppm) or non-functionalized textile (control) are added with an agarose seal (Agarose Sealing Solution (Cat. #786□226) Biosciences) to upper chamber of a transwell employing a COSTAR transwell (Corning, Inc., Corning, N.Y.) with TC treated, PET membrane, diam. 6.5 mm, pore size 8.0 μm sterile cell culture inserts (Sigma-Aldrich No. CLS346). The textile is preferably cotton, and may be other natural, synthetic, or combination textiles. Transwell protocols are as described by Lara, H. H., Ixtepan-Turrent, L., Garza-Trevino, E. N. & Rodriguez-Padilla, C. PVP-coated silver nanoparticles block the transmission of cell-free and cell-associated HIV-1 in human cervical culture. J. Nanobiotechnology 8, 15 (2010). Cell-free virus (2019 BetaCoV/Wuhan/WIV04/2019) [(5×10⁵ TCID₅₀)], are added from the upper chamber through the textile. To evaluate inhibition of the viral infection, efficacies are evaluated by quantification of viral copy numbers in the cell supernatant via quantitative real-time RT-PCR (qRT-PCR) and indicator cells (Vero E6 cells), as described by Wang, M. et al. Remdesivir and chloroquine effectively inhibit the recently emerged novel coronavirus (2019-nCoV) in vitro. Cell Research vol. 30 269-271 (2020), in the lower chamber are cultured and formation of syncytia is monitored for ten days at 37° C. with 5% CO₂.

A positive virus control (textile without functionalization with AgNPs) will produce observable syncytia within seven days of incubation indicative of the presence of infection. A first reading of the plate will be made by day three. Negative control wells will not develop syncytia, which reflect an absence of infection. If either control does not react as expected, the assay is suspect and should be repeated.

In the case of the Vero E6 cells (ATCC (ATCC No. CRL-1586) are maintained in Dulbecco's modification of Eagle medium (DMEM), supplemented with 10% heat-inactivated fetal bovine serum (FBS), filter sterilized. Half of the DMEM/FBS medium is changed for new DMEM with 10% FBS media every three days, and the formation of syncytia is monitored for ten days. The cytopathic effects of the viral infection of Vero 6 cells are analyzed by microscopic assessment of syncytia formation indicative of infection or absence of infection. The percentages of cells showing signs of inhibition of infection transmission is evaluated with respect to the positive control.

What is claimed is:

1. A medical article, comprising:
A medically acceptable substrate suitable for applying topically to an internal or external organ of a body; and
Substantially pure silver nanoparticles having an average diameter between about 1 and 3 nm, the silver nanoparticles being coupled to a surface of the medically acceptable substrate.

2. The medical article of claim 1, wherein the medically acceptable substrate further comprises a silicone substrate

3. The medical article of claim 1, wherein the medically acceptable substrate further comprises a textile substrate.

4. The medical article of claim 2, wherein the substantially pure silver nanoparticles are non-functionalized and ionically coupled to the surface of the silicone substrate.

5. The medical article of claim 3, wherein the substantially pure silver nanoparticles are non-functionalized and ionically coupled to the surface of the textile substrate.

6. The use of the medical article of claim 1 in treating bacterial, fungal, viral infections or combinations thereof.

7. The medical article of claim 1, wherein the substantially pure silver nanoparticles have a net positive charge.

8. A medical wound dressing, comprising a medically acceptable substrate suitable for topical application to a wound, and substantially pure silver nanoparticles a substantially round geometry and an average diameter between about 1 and 3 nm, the silver nanoparticle being applied to a surface of the medically acceptable substrate.

9. The medical wound dressing of claim 8, wherein the medically acceptable substrate further comprises a silicone substrate

10. The medical wound dressing of claim 8, wherein the medically acceptable substrate further comprises a textile substrate.

11. The medical would dressing of claim 9, wherein the substantially pure silver nanoparticles are non-functionalized and ionically coupled to the surface of the silicone substrate.

12. The medical wound dressing of claim 10, wherein the substantially pure silver nanoparticles are non-functionalized and ionically coupled to the surface of the textile substrate.

13. The medical wound dressing of claim 8, wherein the substantially pure silver nanoparticles have a net positive charge.

14. The use of the medical wound dressing of claim 8, in treating bacterial, fungal, viral infections or combinations thereof.

15. Method of treating bacterial, fungal, viral infections or combinations thereof, comprising the steps of:
a. Providing a medically acceptable vehicle adapted to convey an active agent to the situs of infection; and
b. Ionically coupling an active agent consisting essentially of substantially pure silver nanoparticles to the medically acceptable vehicle.

16. The method of treating bacterial, fungal, viral infections or combinations thereof according to claim 15, wherein the step of providing a medically acceptable vehicle further comprises the step of selecting the medically acceptable vehicle from the group of silicone and textile substrates.

17. The method of treating bacterial, fungal, viral infections or combinations thereof according to claim 16, wherein the step of ionically coupling the active agent further comprises the step of ionically coupling the substantially pure silver nanoparticles to the medically acceptable vehicle.

18. The method of claim 15, wherein the substantially pure silver nanoparticles have a net positive charge.
